(19) United States
(12) Patent Application Publication
(10) Pub. No.: US 2015/0177202 A1
(43) Pub. Date: Jun. 25, 2015

(54) ODOR THRESHOLD TEST

(71) Applicant: University of Tennessee Research Foundation, Knoxville, TN (US)

(72) Inventors: Irene Nichols Ozbek, Signal Mountain, TN (US); Manuel Felipe Santiago, Chattanooga, TN (US); Kristin Belle Whitson, Hixson, TN (US); Stefanie Ruth Whitson, Hixson, TN (US); William Arthur Tewalt, Signal Mountain, TN (US); Jessica McKinney, Johnson City, TN (US)

(21) Appl. No.: 14/579,259

(22) Filed: Dec. 22, 2014

Related U.S. Application Data
(60) Provisional application No. 61/919,358, filed on Dec. 20, 2013.

(51) Int. Cl. G01N 33/00

(52) U.S. Cl. CPC G01N 33/0001

(57) ABSTRACT

Provided are methods and kits for determining an odor threshold in a subject. Two or more odors are provided. The subject is exposed to the two or more odors and an odor threshold is determined by detecting a response in the subject to the two or more odors.

### RELATED APPLICATIONS

The presently disclosed subject matter claims the benefit of U.S. Provisional Patent Application Ser. No. 61/919,358, filed Dec. 20, 2013; the disclosure of which is incorporated herein by reference in its entirety.

### TECHNICAL FIELD

The presently disclosed subject matter relates to odor threshold tests. Methods and kits for determining an odor threshold in a subject are also provided.

### BACKGROUND

While numerous diseases and disorders are known to lead to a loss of olfaction, current tests of olfactory sensitivity are not able to use threshold values to establish links between specific ailments and olfactory dysfunction. This is largely due to limitations in methodology and exploratory scope of traditional threshold tests. Accordingly, there is an on-going need for odor threshold tests that can improve performance, such as but not limited to, in the early detection of diseases, and monitoring of progression of and treatment effects on such diseases.

### SUMMARY

Provided in accordance with some embodiments of the presently disclosed subject matter are methods and kits for determining an odor threshold in a subject. In some embodiments, two or more odors or odorant compositions are provided. In some embodiments, the subject is exposed to the two or more odors and an odor threshold is determined by detecting a response in the subject to the two or more odors.

Accordingly, it is an object of the presently disclosed subject matter to provide an odor threshold test. An object of the presently disclosed subject matter having been stated hereinabove, and which is achieved in whole or in part by the presently disclosed subject matter, other objects will become evident as the description proceeds when taken in connection with the accompanying Detailed Description, Examples and Drawings as best described herein below.

### BRIEF DESCRIPTION OF THE DRAWINGS

FIG. 1 is an image of the olfactory epithelium and olfactory bulb of a human subject (image adapted from Murray, 2013).

FIG. 2 is a diagram showing decision making outcomes (adapted from D. Heeger, (1998)).

FIG. 3 is a graph showing a sigmoid curve (s-curve).

FIG. 4 is a graph showing signal plus noise.

FIG. 5 is a graph showing distribution of the decision variable across signal and noise trials (Adapted from Stanislaw & Todorov, (1999)).

FIG. 6 is a bar graph showing demographic data for participants in the study described in the Examples.

FIG. 7 is a structural formula and characteristics chart for ethanol, which is employed as an odorant composition in the Examples.

FIG. 8 is a structural formula and characteristics chart for para-cresol, which is employed as an odorant composition in the Examples.

FIG. 9 is a structural formula and characteristics chart for isoamyl acetate, which is employed as an odorant composition in the Examples.

FIG. 10 is a structural formula and characteristics chart for α-pinene, which is employed as an odorant composition in the Examples.

FIG. 11 is a structural formula and characteristics chart for vanillin, which is employed as an odorant composition in the Examples.

FIG. 12 is a photograph showing an example of the tubes used in the Examples.

FIG. 13 is a graph showing a comparison of estimated vanillin thresholds for subjects with and without asthma.

FIG. 14 is a graph showing a comparison of estimated ethanol thresholds for subjects with and without persistent headaches.

FIG. 15 is a graph showing a comparison of estimated ethanol thresholds between Caucasians and African Americans.

FIG. 16 is a graph showing age vs. response-bias for α-pinene.

FIG. 17 is a graph showing age vs. response bias for para-cresol.

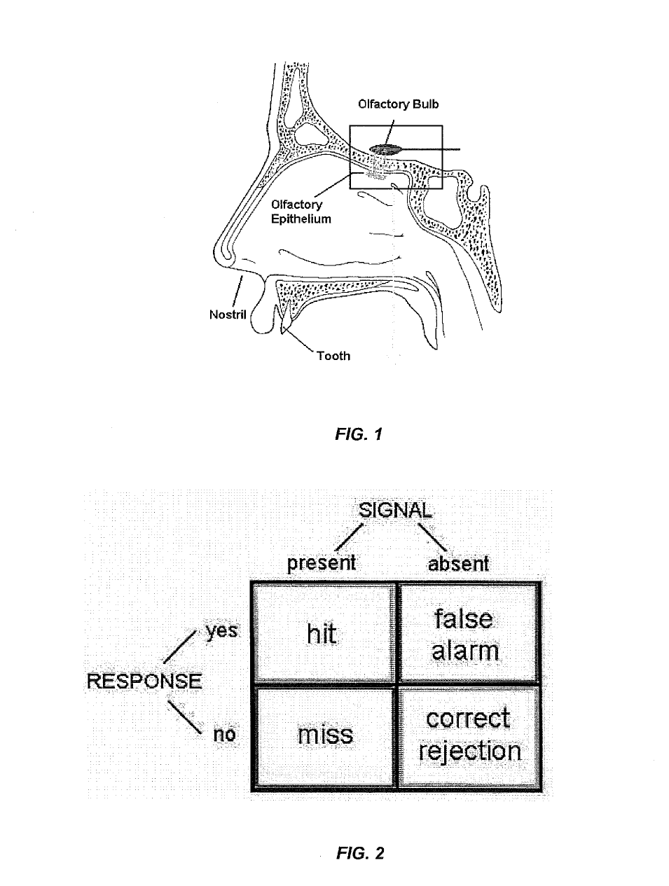

### DETAILED DESCRIPTION

The presently disclosed subject matter relates in some embodiments to an odor threshold test. Representative embodiments are referred to herein as the WUTC (Wheeler UTC) odor threshold test. Threshold tests of olfaction are used to determine the lowest concentration of an odor that a person can detect. Traditional odor threshold tests utilize simple ascending (low to high) or descending (high to low) concentration administrations though some use a stair step method (reversals between low and high concentrations). These tests typically provide the administrator with a single measure of a participant's ability to detect a particular odor.

While numerous diseases and disorders are known to lead to a loss of olfaction, current tests of olfactory sensitivity are not able to use threshold values to establish links between specific ailments and olfactory dysfunction. This is largely due to limitations in methodology and exploratory scope of traditional threshold tests. However, the presently disclosed subject matter at least provides the following representative improvements over prior threshold tests by:
(1) utilizing a diverse odor set, selected based on differences in how they interact with the olfactory system and the chemical properties they possess, which allows for relationships between olfactory deficits to certain odors and specific diseases to be explored;
(2) adopting more closely the guidelines of research methodology, the method of constant stimuli, and signal detection theory. This includes the use of randomized trials, double-blind testing, and multiple presentations of odor concentrations;
(3) providing measures of participant sensitivity, response-bias (with the use of non-stimulus trials), threshold, and inter-rater reliability with a single administration of the test; and
(4) selecting odors and concentrations that do not stimulate the trigeminal (fifth-cranial) nerve allowing for un-confounded assessments of olfactory sensitivity ability to be made.

The presently disclosed subject matter will now be described more fully hereinafter with reference to the accompanying Examples and Figures, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims. Thus, for example, reference to “a compound” includes mixtures of one or more compounds, two or more compounds, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the presently disclosed subject matter.

The term “about”, as used herein when referring to a measurable value such as an amount of weight, molar equivalents, time, temperature, etc. is meant to encompass in one example variations of ±20% or ±10%, in another example ±5%, in another example ±1%, and in yet another example ±0.1% from the specified amount, as such variations are appropriate to perform the disclosed methods.

The term “comprising”, which is synonymous with “including,” “containing,” or “characterized by” is inclusive or open-ended and does not exclude additional, unrecited elements or method steps. “Comprising” is a term of art used in claim language, which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

In some embodiments, the methods and kits disclosed herein can be used with a subject (for example, a living organism, such as a patient). In some embodiments, the subject is a human subject, although it is to be understood that the principles of the presently disclosed subject matter indicate that the presently disclosed subject matter is effective with respect to all vertebrate species, including warm-blooded vertebrates such as birds and mammals, which are intended to be included in the terms “subject” and “patient”. Moreover, a mammal is understood to include any mammalian species for which employing the compositions and methods disclosed herein is desirable, particularly agricultural and domestic mammalian species.

The terms “odorant” and “odorant composition” are used interchangeably herein, and refer to a composition, compound, molecule or other substance or medium from which an odor emanates. Particular odorants can be selected based on guidance set forth here. Five odorants, ethanol, para-cresol, isoamyl acetate, L-α-pinene, and vanillin, are provided herein as representative, non-limiting examples. Other particular examples would include but are not limited to odorants that are hydrophilic, hydrophobic, require protein binding or do not.

### I. GENERAL CONSIDERATIONS

The testing of olfactory sensitivity is something that is seldom used in a clinical setting, yet it has provided very clear and measurable capability as a marker of numerous diseases. In some cases, tests of olfaction can predict future clinical diagnoses of disease better than more expensive and invasive measures. Many patients that are currently experiencing a loss of olfactory sensitivity due to a disease or disorder may not even be aware that any loss has occurred, making regular olfactory testing even more important. However, no olfactory test has yet been able to distinguish between centrally or peripherally caused deficits. Current olfactory threshold tests concentrate solely on how sensitive a participant is to a single odor but have not explored interactions between different types of odors and their ability to be detected by those with certain diseases. Both ‘Sniffin' Sticks’ and the Connecticut Chemosensory Clinical Research Center Test (CCCRC), popularly used tests of olfactory ability, employ threshold tests in their design that only test for the odor n-butanol (Hummel, Sekinger, Wolf, Pauli, & Kobal, 1997). By developing a test with odorants that are selected, for example, based on the diversity of how they interact with the physiology of the olfactory system, the presently disclosed subject matter provides opportunities to determine the pathological cause of the deficit instead of only identifying that a deficit exists.

Causes of Olfactory Dysfunction.

Olfactory impairment can come from a multitude of different sources. In fact, there are more than two hundred known conditions that can lead to changes in chemosensory ability. Table 1 shows that among these conditions, aging, exposure to toxic substances, obstructive nasal and sinus diseases, head trauma, respiratory infection, congenital, and psychiatric disorders are common to result in loss of olfaction, though causes can often be idiopathic.

TABLE 1
Reported Causes of Olfactory Loss (From Walton and Maeso (2012))
Spectrum of Olfactory Loss as Reported at Four Chemosensory Centers

| | Goodspeed | Davidson | Leopold | Heywood |
|---|---|---|---|---|
| | and | and | and | and |
| | Colleagues | Colleagues | Colleagues | Costanzo |
| | (1987)²³⁹* | (1987)²²⁶† | (1987)²²² [symbol] | (1986)¹⁶⁹§ |
| Total no. patients | 441 | 63 | 198 | 133 |
| Etiologic category | | | | |
| (%): | | | | |
| Obstructive nasal | 30 | 33 | 29 | 20 |
| and sinus disease | | | | |
| Post-upper | 19 | 32 | 15 | 17 |
| respiratory | | | | |
| infection | | | | |
| Head trauma | 9 | 10 | 19 | 32 |
| Aging | 0 | 0 | 8 | 6 |
| Congenital | 0 | 5 | 8 | 0 |
| Toxins | 1 | 11 | 3 | 0 |
| Miscellaneous | 14 | 10 | 8 | 16 |
| Idiopathic | 26 | 0 | 10 | 10 |

*Connecticut Chemosensory Clinical Research Center, Farmington, Connecticut.
†Chemosensory Perception Laboratory, University of California, San Diego. [symbol]
Clinical Olfactory Research Center, SUNY Health Science University, Syracuse, New York.
§Smell and Taste Clinic, Medical College of Virginia, Richmond, Virginia.

A person's ability to smell can be directly related to his or her health. Diseases such as Alzheimer's (Dvand, Michaels-Marston, Liu, Pelton, Padilla, & Marder et al. 2000) (Murphey, Gilmore, Seery, Salmon, & Lasker, 1990), Parkinson's (Ross & Abbot, 2005), schizophrenia (Turetsky, Hahn, Borgmann-Winter, & Moberg, 2009) and depression (Negois, Croy, Gerber, Puschmann, Petrowski, Joraschky, & Hummel, 2010) have each been shown to have the reduction in olfactory ability as a comorbidity. Patients with kidney disease undergoing dialysis have also repeatedly been shown to have drastic decreases in their sense of smell. With End Stage Renal Disease (ESRD), patients experience complete anosmia (the inability to detect odors) (Frasnelli, Temmel, Quint, Oberbaur, & Hummel, 2002). In addition, cases of concussion and various types of head trauma have shown to result in altered olfaction (MacCaffrey, 1997). For those who have experienced head injury, tests of olfaction have been shown to be the most sensitive measure of whether any residual neurological impairment exists (Ruff, Ruff, & Wang, 2008).

Additionally, complaints about olfactory ability often arise in patients with depression and schizophrenia as well as disorders characterized by hallucinations. These hallucinations experienced by patients can often be olfactory. This results in patients either believing that an odor is emanating from their own body (intrinsic) or from the environment (extrinsic) (Deems et al., 1991). These chemosensory distortions often lead to decreases in overall quality of life as they can be severe enough to cause disruptions to a patient's daily life and health.

Another known cause of loss of olfactory functionality is nutritional deficiency. In particular, a lack of vitamin A removes the body's ability to repair damage to the nasal epithelium. Duncan and Briggs (1962) have reported that over time, white rats will eventually become anosmic when fed a vitamin A deficient diet. Conversely, the supplementation of vitamin A has been shown to have the ability to partially restore lost olfactory ability (Duncan & Briggs, 1962).

Changes in olfaction emerge in diseases with very dissimilar pathologies. Though some suggest neurological origins of olfactory loss, others point to alterations in the mechanisms of olfactory function. However, little is known about the causes of smell disorders.

Oxidative Stress.

A concept that, in many ways, unifies the theme of olfactory dysfunction and disease is “oxidative stress”. It has been linked to numerous diseases and disorders, as well as aging, and has similarly been shown to be related to olfactory dysfunction.

Oxidative stress occurs when there is an overabundance of free radical oxygen within the body. In part, this is a consequence of natural bodily functions. During the process of respiration, 80 to 90 percent of molecular oxygen (O₂) is transported to cellular tissue and utilized by the mitochondria to create energy in the form of adenine triphosphate (ATP). However, as a natural byproduct of the reaction, small amounts of radical oxygen are produced. This oxygen naturally reacts with a hydrogen that is removed from a helper molecule known as nicotinamide adenine dinucleotide (NADH) during the process of respiration. As a result, water is produced within the cell. However, in addition to water, the oxygen intermediate products superoxide (O₂*⁻), peroxide (O₂⁻), hydrogen peroxide (H₂O₂), and hydroxyl radical (*OH) are also produced (Halliwell, 1992). Radical oxygen within the body is known as a Reactive Oxygen Species (ROS) and these intermediate products are considered the primary forms they take on (Wu et al).

Although the mitochondria are the primary source of natural ROS production in humans (Wu & Cederbaum, 2003), additional sources of ROS include enzymatic processes within the liver and cells. However, not all oxidant stress is caused solely as a natural byproduct of respiration. External factors such as carbon monoxide exposure caused by smoking has been shown to significantly increase levels of oxidative stress (Lopez et al., 2009) as has alcohol abuse (Wu & Cederbaum, 2003). Additional contributors include radiation, UV light, and air pollution as well as certain types of medications. Increasingly, external causes of ROS are being discovered and researched.

ROS pose a danger to people due to the number of normal bodily and cellular processes they take part in and interact with. ROS within the body have the ability to react with various cellular molecules including deoxyribonucleic acid (DNA), proteins, and lipids. Often, ROS cause degradation to these molecules which, in turn, can lead to a change in or disruption of important cellular processes that take place within the body. Additionally, ROS-induced damage to DNA and mitochondrial DNA (mtDNA) has repeatedly been shown to occur. Oxidative damage to mtDNA has been linked to multiple diseases such as neuronal degeneration and cardiovascular disease (Tritschler & Medori, 1993) as well as to increase with aging (Ames, Shigenaga, & Hagen, 1993).

Though oxidative stress damage has displayed the ability to degrade many types of proteins in the body, the protein apolipoprotein E (apoE) has gained interest due to its believed disruption of several important bodily functions such as cognitive processing and immunoregulation (Evola, Hall, Wall, Young, & Grammas, 2010). Deficiencies in apoE have been shown to lead to lower levels of cognitive performance in mice.

Importantly, levels of oxidative stress in the body have been shown to be significantly correlated with numerous diseases and disorders that are characterized by decreased olfactory ability such as Alzheimer's, Parkinson's, and uremia diseases. Higher than normal levels of oxidants “in vivo” have been linked to early onset dementia (Reutens & Sachdev, 2002) and also been shown to precede the principle pathologies of Alzheimer's disease (Perry, Cash, & Smith, 2002) as well as contribute to the creation of senile plaques (Misonou, Morishima-Kawashima, & Ihara, 2000), one of the hallmarks of Alzheimer's disease. Additionally, current research on the subject has found that oxidative stress can lead the creation of inflammatory proteins in the brain (Evola et al., 2010). Inflammation of these proteins causes destabilizing effects on cerebral circulation and blood-brain barrier leakage that can lead to the impairments of learning and memory (Evola et al, 2010). According to Himmelfarb, Stenvinkel, Ikizler, and Hakim (2002), oxidative stress may also be the concept that unifies the prevalence of cardiovascular disease in uremia, a condition marked by a high level of nitrogenous waste in the blood that accompanies renal failure as well as decreased olfactory ability. Those with kidney disease often experience anosmia, or a complete inability to detect the presence of any odor.

The discussed side effects of oxidative stress play a role in olfaction as nearly all diseases with oxidative stress show olfactory impairment as a side-effect. Disruptions to normal bodily function by protein inflammation and/or cellular damage could cause olfactory dysfunction to manifest. Lavin et al. (2013) have shown that, in patients with high levels of inflammation in the olfactory neuroepithelium, decreases in olfactory sensitivity was found to be a better predictor of the inflammation than computed tomography (CT) and endoscopic observation. The cause of this may be linked to the inflammation of Odorant Binding Protein's (OBP's) that exist in the neuroepithelium.

Odorant Binding Proteins.

OBP's play a role in the physiology of olfaction and disrupting their normal function would lead to a decrease in olfactory ability. However, the level of disruption would be dependent on the nature of the odorant that was being smelled as some molecules employ OBP's more than others.

Molecules can be described in terms of their hydrophobicity. This describes the degree to which a molecule is repelled by water. Molecules that are completely hydrophobic are completely insoluble in water, lacking the ability to mix in any proportion. On the other end of the spectrum, completely hydrophilic molecules are miscible, or soluble in water in all proportions. This concept plays a role in the biology of odor detection. Odorant molecules that are hydrophilic are able to pass through the water-soluble membrane of the nasal epithelium and move on to the odorant receptors (Vogt, Prestwich, & Lerner, 1991). However, hydrophobic molecules are unable to pass through the epithelium and require an Odorant Binding Protein to carry them across and to the receptor (Vogt et al., 1991). To do so, the OBP uses a method of facilitated diffusion where it essentially “solubilizes” molecules that are more hydrophobic.

Under situations (such as oxidative stress) where OBP's face inflammation and decreased functionality, a natural interruption of the transport of hydrophobic odors across the epithelium would occur and lead to a lowered ability to detect their presence.

Detection Theory.

Signal Detection Theory (SDT), or simply “Detection Theory”, was developed by David Green and John Swets as a psychophysical approach to the construction and analysis of detection experiments (N. A. Macmillan & Creelman, 2004). Though first developed to deal primarily with tests involving the ability to differentiate auditory stimuli from background noise, SDT has changed over time to incorporate a broader range of analyses. Modern Signal Detection Theory now includes a pool of information that encompasses tests of memory, cognition, and, of course, sensory ability. In fact, this evolution from early SDT has led to the omission of the word “signal”, leaving the collection of methods to be called simply detection theory.

One-Interval Design.

Though detection theory played a role in the development of multiple design strategies for use in measuring sensory performance, in some embodiments an aspect of the present research pertains to one-interval design. This type of design involves the presentation of a single stimulus to a subject on each trial of the test. The stimulus itself has the possibility of being one of a subset of differing stimulus types, depending on the design of the experiment (in accordance with the presently disclosed subject matter, an odor). By utilizing variations of the one-interval design, an experiment can investigate drastically different measures of sensory performance. A use of this design is in measuring discrimination. This describes the ability to distinguish a stimulus from another, different stimulus type. An example would be distinguishing a sweet odor from one that has a pungent scent. There can be two representative types of discrimination tasks, the first of which is termed “detection”. In SDT, detection task trials contain a stimulus as well as a null-stimulus and the participant must determine which they are currently being presented with. However, a discrimination task that does not contain a null-stimulus produces a performance measure termed “recognition” as a participant attempts to recognize which of multiple stimuli is being presented. Finally, one-interval experiments can take the form of measuring the ability to identify/classify stimuli. In this task, stimuli differ from each other in one characteristic which is then “identified” by the participant upon the stimulus presentation.

One-interval designs can be used for diverse applications depending on the types and number of stimuli classes used in the experiment. While this type of experiment involves the presentation of a single stimulus for each trial, there are other methods of evaluating discrimination available. In particular, a popular alternative choice to the one-interval design is the “Two-alternative forced choice” (2AFC) test (N. A. Macmillan & Creelman, 2004). While still a test of discrimination, participants in a 2AFC test are presented with two stimuli per trial that are randomly separated by time or position. Though it can be viewed as an extended one-interval design, this type of test is considerably different in that a participant is not being asked to discriminate between stimulus type, but instead by stimulus order. When increasing beyond the presentation of two stimuli in a single discrimination trial, the experimental design adopts the name or the m-alternative forced choice (mAFC) where the value of m represents the number of choices presented in each trial (N. A. Macmillan & Creelman, 2004).

Yes-No Trial.

For the aforementioned one-interval design that is set up for the purpose of measuring stimulus detection ability, only one of two responses is possible for each trial. These responses are “yes” and “no”. When responding to each trial in a detection experiment, a participant's answer can ultimately be categorized as one of four types of events. These include the hit, miss, false rejection, and correct rejection.

The goal of a yes-no experiment is two-fold: 1) to compare participant responses to the type/level/degree of the stimulus, and 2) determine the amount of bias present. The first of these goals focuses on what is termed sensitivity, or the measurement of a participant's ability to discriminate between stimuli. In terms of detection, a person with high sensitivity has a greater ability to detect stimuli than one with poorer sensitivity. The second of these goals, determination of bias, involves measuring a participant's inclination to answer “yes”.

Yes-no experimental designs can be susceptible to the effects of participant response-bias. Other experimental methods of measuring detection, such as the 2AFC and mAFC designs, have more recently become widely adopted due to their minimal response bias. However, these paradigms often take considerably longer to administer and have been found to be more statistically biased than yes-no experimental designs (Kershaw, 1985). This is due to 2AFC and mAFC having truncated ranges in their psychometric functions compared to the maximized range of a yes-no design.

Threshold.

In addition to measures of sensitivity and response-bias, one-interval detection experiments also provide the ability to estimate a “threshold”, or the “magnitude of the weakest detectable stimulus” (N. A. Macmillan & Creelman, 2004). Estimations of stimulus thresholds can be produced in a variety of ways and this variety is dependent in part on the method of stimulus administration used by the researcher. The order of stimulus presentation for each trial of a test often takes one of four possible forms; 1) Increase of stimulus level from lowest to highest, 2) Decrease stimulus level from highest to lowest, 3) a type of stair-case method that alternates between a high and low stimulus magnitude, and 4) randomized stimulus level.

The “threshold” is often defined as a stimulus which is detected, or responded yes to, on 50% of the trials given. One method for determining an individual's threshold for a stimulus is the use of logistic regression represented by the function (N. A. Macmillan & Creelman, 2004):

$$λ ( x ) = \frac{{e}^{x - μ}}{{[ 1 + {e}^{x - μ} ]}^{2}}$$

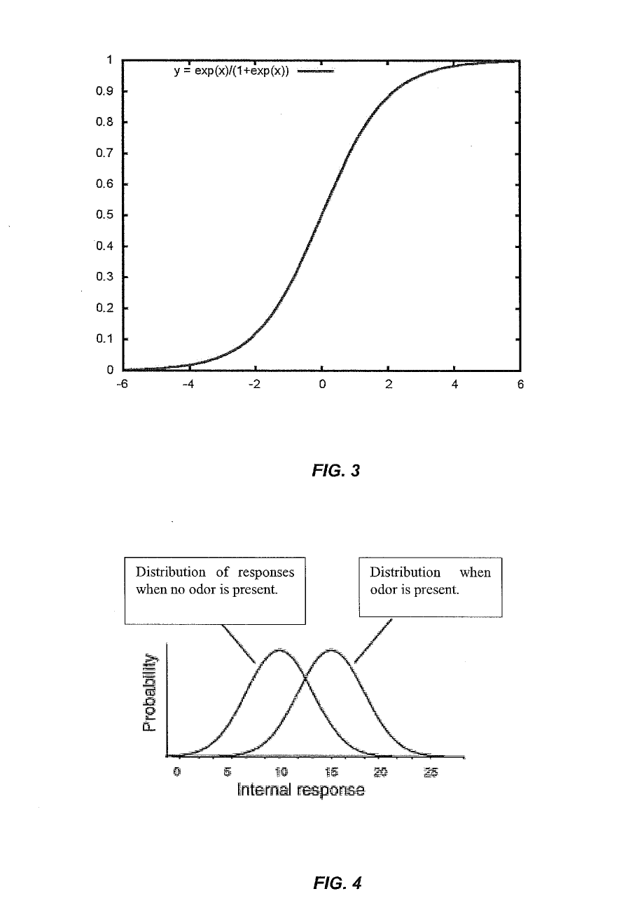

The use of the function can be used to determine predicted probabilities for each level of a stimuli, which can then be used to find an estimated threshold value. The threshold is the stimulus deemed to have a value that corresponds to a p-value of 0.5 on the sigmoid curve (s-curve). A visualization of the s-curve is provided in FIG. 3. When comparing thresholds, lower values indicate a better ability to detect stimuli.

A benefit of using a threshold model is that, unlike measures of sensitivity, it gives an actual calculated level of the lowest stimulus detectable.

Noise.

The term “noise” represents anything that compromises the ability to detect a stimulus by introducing a level of uncertainty on whether or not the stimulus is present (N. A. Macmillan & Creelman, 2004). Two types of noise can ultimately contribute to a level of uncertainty. These are internal and external noise. External noise can come from many different sources depending on the nature of the discrimination test but are often factors that exist in the environment. Examples of noise could be static in a test of auditory detection or an odor in the testing area of an olfactory test. Internal noise, however, is a result of both cognitive and sensory components within the participant that lead to uncertainty or error.

In detection tests, a stimulus trial is employed to represent stimuli plus noise and a null-stimulus trial would be the occurrence of only noise. FIG. 4 shows how the presence of noise can result in a lower signal to noise ratio.

Due to the existence of noise in all trials of a test, efforts are employed to minimize any noise present and to attempt to keep testing conditions consistent across all trials and participants. For any test of stimulus detection, it is often the goal of the researcher to attempt to create a noise-free environment that results in the greatest signal-to-noise ratio possible, thereby enhancing the ability to measure signal discriminability.

Measures.

There are multiple measures that can be used to describe olfactory ability.

Sensitivity.

In SDT, several statistics can be used to describe different facets of a participant's detection ability. The first, a sensitivity measure index known as d′, is considered to be a pure measure of sensitivity that is unaffected by any response bias as long as the signal and noise distributions are both normal (Swets, 1986). The calculation of d′ provides a measurement of the difference between the signal and noise means in standard deviation units. Therefore, a d′ value of zero (0) indicates an inability to distinguish between the signal and noise trials and positive values represent increasing levels of sensitivity. Participants that are unable to discriminate between stimuli and false positive and obtain identical hit (H) and false positive (F) rates, H=F, will therefore obtain a d′ equal to zero. However, problems arise when H=1.0 as this causes d′ values to become infinite, regardless of the proportion of false-positives they had. Fortunately, there are multiple methods of fixing hit-and-false positive rates to avoid this. One method, termed a “logilinear” approach, involves simply converting H and F proportions from values of 0 and 1 by adding 0.5 to both the amount of hits and false alarms and adding 1 to the total number of signal and noise trials (Hautus, 1995; Miller, 1996). Another approach involves the adjustment of extreme rates with the following conversions where n is the number of trials (N. Macmillan & Kaplan, 1985):

Rate of 0→0.5/n

Rate of 1.0→(n−0.5)/n

Though this method of adjusting extreme rates is an accepted tactic, it is believed to yield sensitivity measures that are more biased than those of a logilinear approach (Miller, 1996). Additional solutions to the issue of extreme values in generating a d′ measure of sensitivity exist, however their usefulness is highly debated as they involve combining data sets or the reliance on alternative statistical measures.

Another measure of sensitivity known as A′ (A-prime) is also widely used and accepted as a measure of sensitivity (N. A. Macmillan & Creelman, 1996). A popular reason for this is its non-parametric nature, meaning that there are no assumptions made about statistical parameters. A measure of this kind is often considered to be more statistically “robust”, meaning that it performs well in a variety of probability distributions. However, a downside to A′ is that it might be necessary to employ more statistical power than d′ to provide an accurate measurement of sensitivity.

Response Bias.

In a yes-no experimental design, there can be a risk of participant response-bias, or the tendency to say “yes”. The standard measurement of response bias is computed as β and is based on a likelihood ratio of either a “yes” or “no” response on a signal trial. A value of β=1 corresponds to a participant being effectively “neutral” in the tendency to respond either yes or no to a given trial. Those who have a tendency to respond yes have β values less than 1 whereas a value greater than 1 indicates a tendency to respond no. Being based on likelihood ratio, values of β are often represented instead by ln(β).

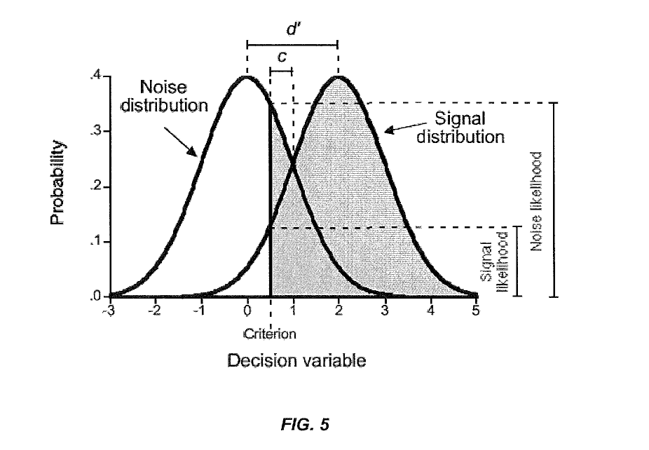

Though β has been popularly used to measure response bias, there is growing support for the use of the statistic, criterion (c) (Banks, 1970); (Neil A. Macmillan & Creelman, 1990). In signal detection theory, c is the average of the z scores for both the hit and false alarm rates multiplied by negative one. The range of possible values for the c statistic extends from c=−2.33 to c=2.33. In a case where the false-alarm rate is larger than the miss rate, the criterion value will be negative. Negative values of c indicate that there is a bias towards responding “yes” during a trial. This also means that when values of c become smaller, there is an increase in the tendency of a participant to make “yes” responses. Alternatively, positive values of c correspond to a response-bias that is slanted towards responding “no”. The primary benefit of using the statistic c instead of 13 as the primary measure of response-bias is that c is unaffected by changes in d′ (Ingham, 1970). Visualization of the measures d′ and c, as they relate to the signal and noise distributions in a detection trial, can be seen in FIG. 5.

Reliability.

Apart from sensitivity and response-bias, the fundamental measurements of signal detection theory, researchers are often concerned with the consistency of a measure. In psychometrics, estimates of consistency describe a measure's “reliability”. If a detection test were to produce stable results across multiple trials to the same participant, it could be said that the test exhibits high reliability. Nunnally (1967) defined reliability as “the extent to which measurements are repeatable and that any random influence which tends to make measurements different from occasion to occasion is a source of measurement error”. For a one-interval, yes-no detection test, a test-retest reliability measure can be made by measuring the consistency of participant sensitivity, bias, and responses across multiple administrations of the same test. Alternatively, a detection test can be divided into two equivalent halves and a measure of consistency can be assessed between them. Reliability of this type is known as “split-half” as it involves the comparison of multiple, parallel forms of a test that are administered within the trials of a single test. When splitting a test in this way, it is incredibly important to attempt to create test halves that are as similar as possible.

One of the most widely used and important measures of reliability is known as the “coefficient alpha” or “Cronbach's alpha” (Cronbach, 1951). According to Cronbach (1951), alpha is the mean of all the split-half reliabilities. Though the essence of the theory behind Cronbach's alpha will not be described in depth here, its description is often one of a coefficient of equivalence. Acceptable values of alpha are displayed in Table 2.

TABLE 2
Values of Cronbach's alpha

| Cronbach's | Internal |
|---|---|
| α ≧ .9 | Excellent |
| .9 > α ≧ .8 | Good |
| .8 > α ≧ .8 | Acceptable |
| .7 > α ≧ .6 | Questionable |
| .6 > α ≧ .5 | Poor |
| .5 > α | Unacceptable |

Note:
Adapted from Darren & Mallery, (2003)

Odor Threshold Tests.

Currently, there are two odor threshold tests that are popularly used in clinical settings. The first of these is ‘Sniffin' Sticks’, a chemosensory test that uses pen-like sticks to dispense an odor to participants during administrations. The threshold portion of the ‘Sniffin' Sticks’ test includes the presentation of n-butanol filled dispensers with various concentrations that are administered in a single staircase method. Each odor pen uses a propylene glycol solvent. During administration, the ‘Sniffin’ Stick is placed approximately 2 cm from the participant's nose for around 3 seconds. Testing follows a triple-forced-choice, single staircase paradigm in which subjects are presented with a single odor concentration and two blanks and asked to respond yes or no as to whether they detect an odor. Upon the correct detection of the signal in two successive trials, the staircase is reversed for a total of seven reversals and the geometric mean of the last four is calculated and deemed the participant's “threshold” (Hummel et al., 1997). This threshold value is given as a test “score” as individual dispenser concentrations are not provided. However, the ‘Sniffin' Sticks’ test uses a top concentration of 4% n-butanol and a dilution factor of 1:2 (Hummel et al., 1997).

The second test commonly used to determine participants' odor threshold is the Connecticut Chemosensory Clinical Research Center Test (CCCRC). Similarly to the ‘Sniffin' Sticks’ test, the CCCRC uses n-butanol as its primary odorant. The odor is dispensed with the use of plastic squeeze bottles. The highest concentration of n-butanol used in the series is 4% in water with 11 additional geometric dilutions following a ratio of 1:3. Participants are tested with a 2AFC ascending model where each trial contains one signal and one null-stimulus in which subjects attempt to identify the bottle containing the odorant. In the CCCRC, the threshold value is given as the concentration in which the participant was able to succeed in identifying the signal and the 5 successive trials that preceded it.

Problematic for each of the two tests is the use of n-butanol as the single odorant being tested. According to (Brand, 2006), n-butanol produces more activation of the trigeminal nerve than molecules that have a larger olfactory component, such as floral or sweet odors. Activating the trigeminal nerve (or 5^{th} cranial nerve) results in feelings of pain that can be detected even in the absence of odor detection. Though the molecular concentration of n-butanol in an odor trial also plays a large part in whether it leads to stimulation of the trigeminal system, irritation has been found to be caused by concentrations of approximately 200 ppm, considerably less than what is found in the CCCRC and ‘Sniffin Sticks’ tests. Instead of isolating the olfactory system, this nerve activation can lead to changes in olfactory information processing (W. Silver, 1991).

Though the CCCRC and ‘Sniffin' Sticks’ threshold tests both employ the use of blanks in determining an individual's threshold, they are not used to measure additional statistics such as participant response-bias or sensitivity.

### II. METHODS AND KITS

Disclosed herein in some embodiments are methods of and kits for measuring olfactory sensitivity. In some embodiments the presently disclosed odor threshold tests are inexpensive and non-invasive. They set standards that prior instruments lack and in so doing, improve the specificity, sensitivity, reliability and validity of olfactory threshold measurement. For example, randomization of odorants and concentrations, inclusion of blanks, double blind administration, selection of odorants relevant to particular disease states for early detection, and monitoring of progression of and treatment effects of those particular diseases are aspects of the presently disclosed subject matter. The presently disclosed tests can be employed for medical purposes in terms of detection of a biomarker of which the individual may be unaware. Thus, like blood pressure measurement, the presently disclosed tests allow for measurement of one or more biological functions.

Other available threshold tests use the staircase method of administration of odorants with concentrations of the odorants progressively administered until an odor is detected. Habituation, subject bias, and subject consistency in the context of random presentation are not addressed or measured with the staircase method. The WUTC thus provides an assessment procedure that can be used in terms of assessment of additional factors other than presence and severity of disease state. Other factors include insurance, disability, workman comprehensive coverage claims and law suits.

The presently disclosed tests of olfactory ability employ in some embodiments the idea that deficits in olfactory ability are not necessarily generalizable to all odors. Though numerous diseases and disorders have been shown to lead to a loss of olfaction, tests of olfactory sensitivity have been limited to performance detecting a single odor. In some embodiments, the presently disclosed tests employ two or more odors, such as but not limited to five odors, that were selected based on differences in how they interact with the olfactory system and the chemical properties they possess. By utilizing a diverse odor profile, relationships between olfactory deficits to certain odors and specific diseases can be explored. In some embodiments, the presently disclosed tests also employ randomized, multiple presentation of odorants along with null-stimulus trials. Using this methodology, statistical measures of participant sensitivity, response-bias, threshold, and inter-rater reliability can be calculated with a single administration of the test. A study, including thirty three (N=33) participants, was conducted, as disclosed herein below. Subject demographic data was also collected in order to conduct exploratory analyses and aid in the further development of the test. Representative reasoning and methodology are discussed along with the analyses of the subject data. Representative aspects of the results of this study suggest that certain ailments do not have significant olfactory deficits to all odorants, but to particular odor molecules. Representative principles behind the development of the presently disclosed tests provide for understanding of links between olfaction and disease and an increase in the value of examining olfactory ability in a clinical setting.

In accordance with some embodiments of the presently disclosed subject matter, a method of detecting and/or determining an odor threshold in a subject is provided. In some embodiments, the method comprises providing two or more odors; exposing the two or more odors to a subject; and determining an odor threshold by detecting a response in the subject to the two or more odors. In some embodiments, the two or more odors is two, three, four, five or more odors. In some embodiments, the odors are selected based on differences in how they interact with the olfactory system of the subject and/or chemical properties they possess. In some embodiments, the odors differ in a characteristic selected from the group including but not limited to a descriptive quality, a molecular classification of odorant, a level of hydrophobicity of odorant, and any combination thereof.

In accordance with some embodiments of the presently disclosed subject matter, the odors or odorant compositions are presented to the subject in a random sequence, in one or more different concentrations, or combinations thereof. Representative concentrations and dilution strategies for representative non-limiting odorant compositions are provided in the Examples presented herein below. Other odorant compositions, concentrations, and dilatation strategies would become apparent to one of ordinary skill in the art upon a review of the present disclosure. In some embodiments, the presently disclosed subject matter employs randomized, multiple presentations of odorants, optionally with null-stimulus trials. In some embodiments, the presently disclosed subject matter employs a double-blind design in exposing the two or more odors to the subject. In some embodiments, the exposing of each odor to the subject is repeated.

In accordance with some embodiments of the presently disclosed subject matter, a conclusion about the subject is drawn, based on the odor threshold that is determined. In some embodiments, the conclusion relates to a demographic characteristic of the subject. Representative, non-limiting demographic characteristics are provided in the Examples presented herein below. Other demographic characteristics would become apparent to one of ordinary skill in the art upon a review of the present disclosure.

In some embodiments, the conclusion relates to a medical condition of the subject. Representative aspects of the medical conditions are disclosed herein and include but are not limited to the presence of the medical condition, the progression of the medical condition, and/or the response to treatment by a medical condition. Representative medical conditions include are not limited to medical conditions involving oxidative stress. Particular medical conditions themselves include are not limited to asthma, Alzheimer's Disease, Parkinson's Disease, depression, kidney disease (such as but not limited to kidney disease undergoing dialysis), End Stage Renal Disease (ESRD), concussion and various types of head trauma, schizophrenia, disorders characterized by hallucinations, and nutritional deficiency.

In accordance with some embodiments of the presently disclosed subject matter, a kit for determining an odor threshold in a subject is provided. In some embodiments, the kit comprises two or more containers, each container containing a different odorant composition; and instructions for determining an odor threshold by detecting a response in the subject to the two or more odors. In some embodiments, the kit comprises two, three, four, five or more containers. In some embodiments, each container contains a different odorant composition. In some embodiments, the odors are selected based on differences in how they interact with the olfactory system of the subject and/or chemical properties they possess. In some embodiments, the odors differ in a characteristic selected from the group including but not limited to a descriptive quality, a molecular classification of odorant, a level of hydrophobicity of odorant, and any combination thereof.

In some embodiments, the instructions included provide for presenting the two or more odors to the subject in a random sequence. In some embodiments, the instructions include comprising employing randomized, multiple presentations of odorants, optionally with null-stimulus trials. In some embodiments, the instructions include employing a double-blind design in exposing the two or more odors to the subject. In some embodiments, the instructions include repeating the exposing of each odor to the subject.

In some embodiments, the instructions include drawing a conclusion about the subject based on the odor threshold. Thus, in accordance with some embodiments of the presently disclosed subject matter, a conclusion about the subject is drawn, based on the odor threshold that is determined. In some embodiments, the conclusion relates to a demographic characteristic of the subject. Representative, non-limiting demographic characteristics are provided in the Examples presented herein below. Other demographic characteristics would become apparent to one of ordinary skill in the art upon a review of the present disclosure.

In some embodiments, the conclusion relates to a medical condition of the subject. Representative aspects of the medical conditions are disclosed herein and include but are not limited to the presence of the medical condition, the progression of the medical condition, and/or the response to treatment by a medical condition. Representative medical conditions include are not limited to medical conditions involving oxidative stress. Particular medical conditions themselves include are not limited to asthma, Alzheimer's Disease, Parkinson's Disease, depression, kidney disease (such as but not limited to kidney disease undergoing dialysis), End Stage Renal Disease (ESRD), concussion and various types of head trauma, schizophrenia, disorders characterized by hallucinations, and nutritional deficiency.

### III. EXAMPLES

The following Examples have been included to illustrate modes of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

The present study involved the creation and study of a new test of olfactory ability, deemed the Wheeler University of Tennessee, Chattanooga (WUTC) Odor Threshold Test. This test was developed building upon the methodology behind signal detection theory as it allowed for multiple measures of olfactory ability to be calculated from a single test. These measures are sensitivity, response-bias, and threshold. Unlike currently available threshold tests, the WUTC Odor Threshold Test utilizes a randomized, multiple odor administration along with the presentation of blank concentrations.

By using multiple odors, the WUTC can look for relationships between odor property and its ability to be detected by those with different diseases or disorders. The odors used were selected based on the diverse properties they possess. Odors differ in their descriptive quality (sweet, pungent, etc.) as well as their molecular classification. Additionally, odorants have varying levels of hydrophobicity, which lead to different Odorant Binding Protein (OBP) usage to be detected. Due to the inflammation and degradation of proteins observed in many diseases, the lowering of the detectability of odors requiring OBP's may be seen. By presenting the odors to participants in random sequence, an attempt can be made to decrease levels of olfactory fatigue to individual odors. Finally, the WUTC adheres to research methodology by employing a double-blind design along with multiple administrations of each odor to participants, allowing for inter-rater reliability to be determined for each odor. These reliability measures can lead to additional comparisons to be made with demographic data as well as aid in the selection of odors that have higher reliability between multiple administrations.

Participants.

A total of thirty three participants (N=33), collected from the UTC campus, were administered the WUTC threshold test. Subject ages ranged from 18 to 46 years old (M=23.69, SD=7.917) for the 32 participants who provided their age. The subjects included 12 (36.4%) male and 21 (63.6%) female. Out of this sample, 23 (69.7%) of tested individuals were Caucasian and 10 (30.3%) were African American. Information on current education was collected from each subject and is shown in Table 3.

TABLE 3
Participant Data on Current Educational Status

| | | | Valid | Cumulative |
|---|---|---|---|---|
| College Education | Frequency | Percent | Percent | Percent |
| Freshman | 9 | 27.3 | 27.3 | 27.3 |
| Sophomore | 6 | 18.2 | 18.2 | 45.5 |
| Junior | 11 | 33.3 | 33.3 | 78.8 |
| Senior | 3 | 9.1 | 9.1 | 87.9 |
| Five or more years | 4 | 12.1 | 12.1 | 100 |
| Total | 33 | 100 | 100 | |

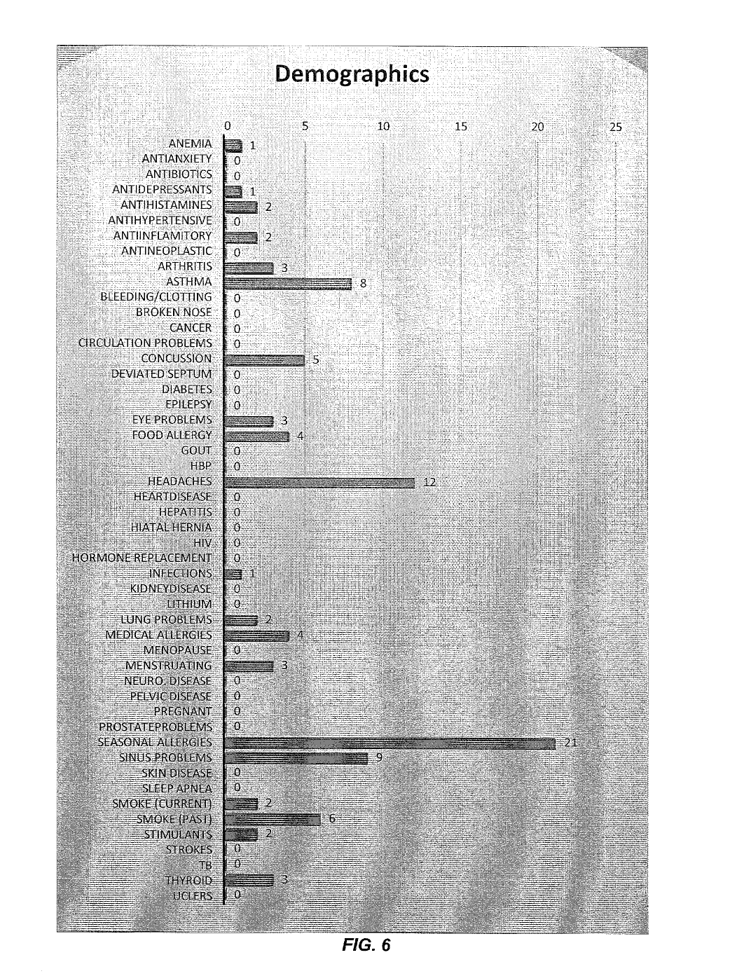

Participants were also asked to complete a demographic form (see below) with detailed questions about their personal health. The form included questions about smoking habits, any current diseases or disorders, medications, menstruation, and pregnancy (see FIG. 6). The most frequently reported demographic information between all subjects were seasonal allergies (N=21, 63.6%), persistent headaches (N=12, 36.4%), sinus problems (N=9, 27.3%), and asthma (N=8, 24.2%). A total of 8 participants (24.2%) circled “yes” to smoking on the demographic form though six of those had not smoked for greater than one month.

Materials and Procedure.

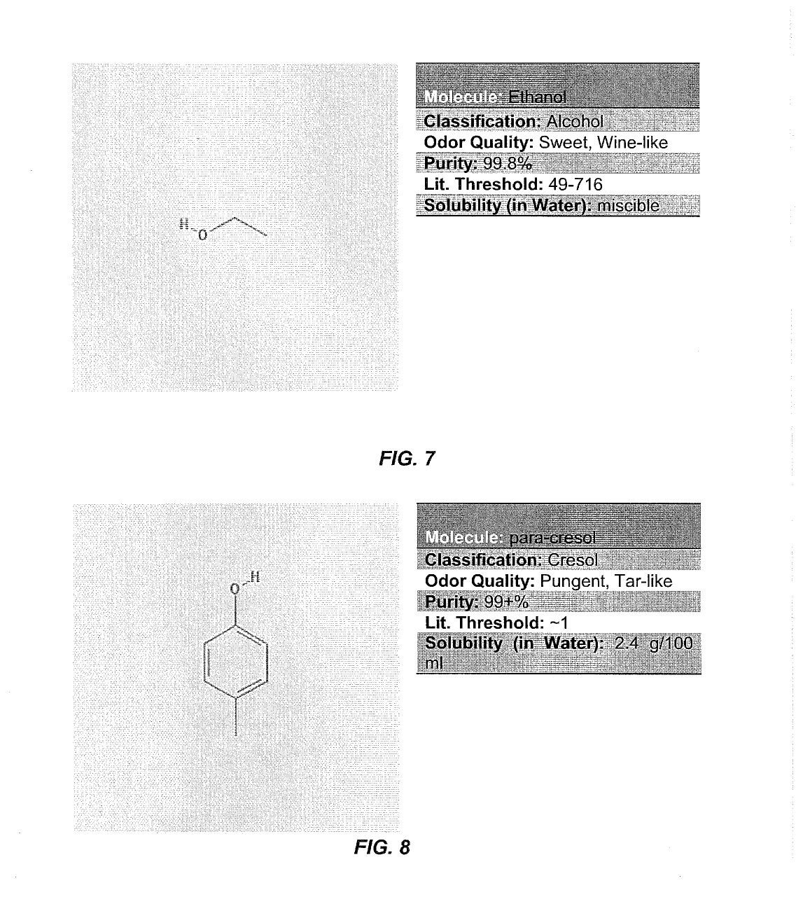

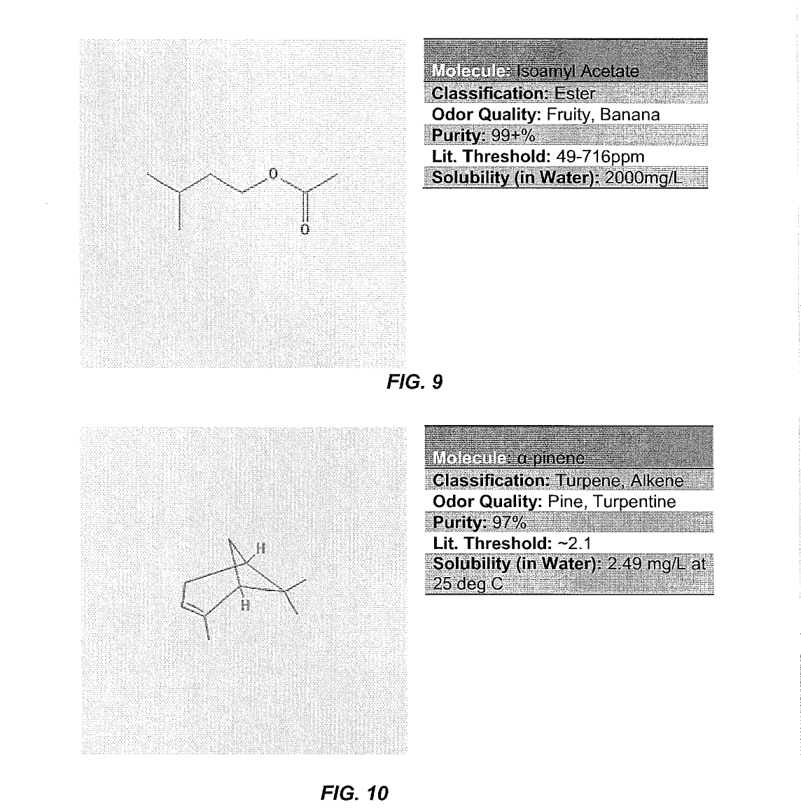

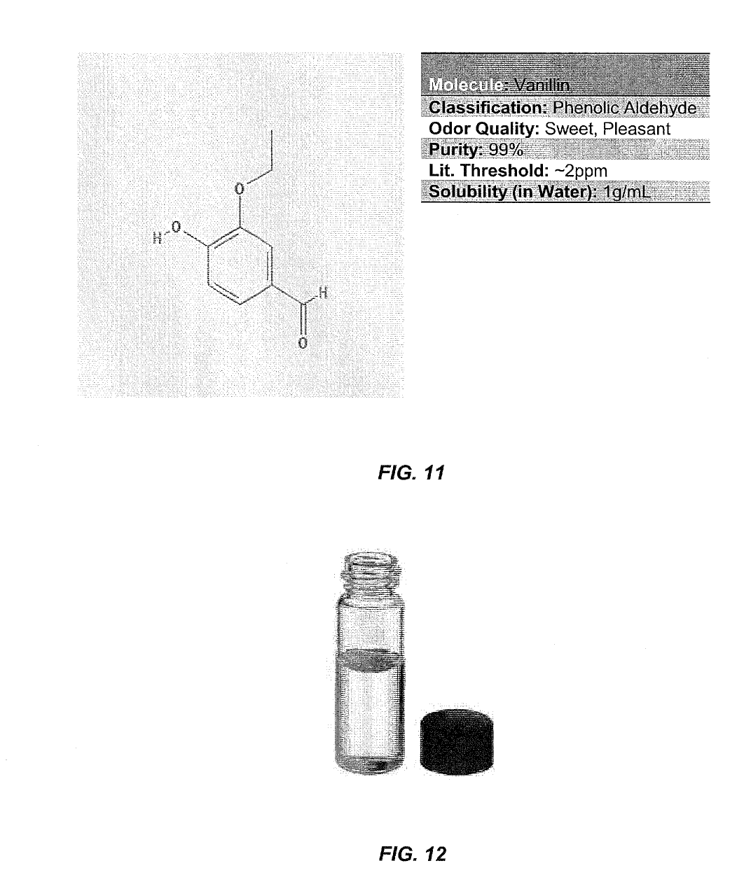

In creating the test, the five odorants ethanol, para-cresol, isoamyl acetate, L-α-pinene, and vanillin were chosen because of various factors. Properties for each odor molecule are included in the same order as the FIGS. 7 through 11. First, the two molecules ethanol and α-pinene were used based on their hydrophobicity characteristic and need for Odorant Binding Protein (OBP) interaction in crossing the nasal epithelium. Their inclusion allows for the ability to determine whether there is any damage to these proteins present in an individual.

Ethanol is completely miscible, meaning that it is completely mixable in water in all proportions. This hydrophilic nature allows it to cross the water-soluble membrane of the epithelium and reach the odor receptors. Research conducted by W. L. Silver, Mason, Russell, Michael, & Smeraski, 1986 determined that the degree to which an alcohol is an irritant is directly related to the length of its carbon chain with increasing irritability as the number of carbons increased. With only two carbons, ethanol does not produce irritation in the trigeminal nerve until it is encountered in concentrations over 1000 ppm. Because methanol (an alcohol containing only one carbon) has been shown to have wildly fluctuating threshold values based on purity, ethanol was deemed the more suitable choice for use in the WUTC.

L-α-pinene, unlike ethanol, is extremely hydrophobic. This results in a need for an OBP to transport the molecule across the water-soluble membrane of the nasal epithelium (Pevsner & Snyder). Though pinene is a known irritant and usually stimulates the trigeminal nerve, it has been shown that the stereospecificity of the molecule plays a large role in its potency (Kasanen et al., 1998) with L-α-pinene being nearly inactive as an irritant.

The odorant vanillin was chosen to be used in the threshold test due to the known differences in ways that it is processed by infants. Vanillin has been shown to significantly prevent apnea in premature newborn infants (Edraki et al., 2013). Being one of the first odors recognizable and preferred by infants, vanillin detectability may prove to be related to infant and childhood development.

The final two odors are isoamyl acetate and para-cresol. Isoamyl acetate, which has the fruity smell of bananas is vastly different from the pungent, tar-like odor of para-cresol. Additionally, para-cresol has been identified as a uremic toxin (Vanholder et al., 2003). By adding these final two odors, the odorant quality profile of the test is very diverse. Also diverse is the compound class of the molecules with the odors containing varied functional groups and structures. The inclusion of this variety of molecules in a single test can allow for the exploration of olfactory deficits to specific odor properties to be explored.

Odorant Dilutions.

To make the test, odorant molecules were first dissolved in a purified H₂O solvent following their individual levels of solubility to create standard solutions. These standards were the highest concentration for each odorant and the base from which all successive dilutions were made. Liquid odorants ethanol, pinene, and isoamyl acetate were diluted by volume whereas para-cresol and vanillin were diluted by mass. Each standard was rounded to the nearest μL. A total of nine concentrations were made from each standard solution and diluted at a ratio of 1:2. The highest concentration for each odor as well as concentration ranges were chosen based on literature threshold values and a small, preliminary testing period. Solutions (10 mL) of each odorant were diluted and contained in sterilized and dried glass vials with black, screw-top lids (see FIG. 12).

Each vial was left unmarked and liquids were visually clear and characterless. Blanks were made using 10 mL of the same purified H₂O used as a solvent for the other odorants. The final test contained 45 vials with odorant concentrations and nine blanks for a total of 54 vials. Tests were remade after either one month or ten administrations had been reached to avoid any amount of loss of odor strength that could result from extended shelf time or exposure to air during administrations. Before reproducing the test, vials underwent sterilization and drying procedures.

To address the shelf-life and possible diminishing of strength of odor concentrations over time, the WUTC was remade approximately after ˜1 mo. of use or 10 administrations.

Administration.

Each subject was instructed to complete the provided demographic form (see below). These preliminary steps were helpful not only in the collection of data for this study, but also in allowing subjects time to adapt to any olfactory stimuli that may have been present in the testing location, despite efforts to minimize such stimuli. Each participant was then seated in a cushioned, high backed chair facing away from a table where each testing vial was placed. In a brief tutorial to the test, subjects were instructed that they would be presented with a number of vials, some containing odors and some not, one at a time. Subjects were then told that they would only be required to smell the contents of the vial and verbally give a “yes” or “no” answer as to whether they detected anything. Continuing the tutorial, a capped tube was held by tongs and placed approximately 1 cm below the center of the participant's nose, demonstrating that this would allow for both nostrils to have equal opportunity to smell the liquid inside. Once the subject felt comfortable with the instructions presented in this tutorial, the actual test was started.

Following a randomly generated number sequence for each subject, a seated test administrator would select the correspondingly numbered test vial for each trial, place it in tongs, and hand it to a second administrator that, like the participant, was facing away from the administration table. The second administrator would then remove the top to the vial and place the tube under the subject's nose, as previously demonstrated in the tutorial. The “yes” or “no” response given by the subject was recorded by the first administrator and the vial and tongs were returned to him/her by the second administrator. This procedure was repeated for each of the odorant tubes in the test with each tube presented to the participant twice. Throughout the entirety of the testing, only the seated administrator was aware of the vial being presented for each trial as well as the number of trials remaining in the test. By doing so, the test followed a double-blind procedure. Both administrators utilized non-latex, medical gloves during each test administration to keep the vials as clean as possible and free from oils or residue that may have been present on the administrator's hands. At the conclusion of the test, participants were debriefed and any questions they may have had were answered. Administration time varied depending largely on subject response time, normally taking between 35 and 45 minutes from the time they entered the room until they were finished and departed.

Analysis.

Multiple statistical tests were used to define the value of the WUTC as a measure of olfactory sensitivity. Unlike other olfactory sensitivity testing methods, the inclusion of multiple administrations and a random presentation of trials in the WUTC garners a much deeper pool of statistical information that permits for a wider breadth of relationships to be explored. This allows the WUTC to describe each participant's olfactory ability in four different ways for each odor. These comprise: 1) Sensitivity, 2) Response-bias, 3) Threshold, and 4) Inter-rater Reliability.

### Example 1

### Sensitivity

To measure olfactory sensitivity, the standard SDT measure d′ was calculated. This statistic gave clear representation of the differences in sensitivity to different odors participants had due to values being on the same scale. The index was calculated as follows (Neil A. Macmillan, 1993):

d′=z(H)−z(F)

The calculation for the d′ sensitivity measure involves z-transformations of both the hit and false alarm rates, converting them to z-scores. The resulting difference between these z-scores then becomes the measure of accuracy, d′. Values of the d′ statistic range from zero to 4.65, what is considered to be its “ceiling” (N. A. Macmillan & Creelman, 2004), with low values corresponding to lower sensitivity and higher values to high sensitivity to a stimulus. In order to compute values of d′ in the presence of Hit or False Alarm rates being equal to 1 or 0, a logilinear (Miller, 1996) approach was used.

In addition to d′, the sensitivity index A′ was calculated due to its non-parametric nature. A′ was calculated with the equation (Snodgrass & Corwin, 1988):

$${A}^{′} = \{ \begin{matrix}.5 + \frac{( H - F ) ( 1 + H - F )}{4 H ( 1 - F )} & when H \geq F \\ .5 - \frac{( F - H ) ( 1 + F - H )}{4 F ( 1 - H )} & when H < F\end{matrix} .$$

Values for A′ range from 0 to 1 with a value of 0.5 indicating that the signal trials were unable to be distinguished from noise.

Both d′ and A′ were calculated from a combination of all participant data (n=33). This provided mean sensitivity statistics for each concentration of the five odors of the test. Therefore, there were a total of forty-five calculated values of both d′ and A′ (nine concentrations of each of the five odors).

### Example 2

### Response-Bias

Response-bias was computed as a way to measure the tendency of participants to answer either “yes” or “no” during both signal and noise trials. The value c is defined in the equation (Neil A. Macmillan, 1993):

$$c = - \frac{1}{2} [ z ( H ) + z ( F ) ]$$

The statistic c was used in place of the standard response-bias measure β due to its independence from changes in d′. Measurements of response-bias were calculated for each participant and also for all participant data combined.

### Example 3

### Threshold

The estimated odor threshold of each participant for the individual odors of vanillin, pinene, ethanol, isoamyl acetate, and para-cresol were also obtained. To calculate the threshold values, yes/no responses were analyzed using logistic regression and a set of predicted values were generated based on those responses. The estimated threshold value was designated as the odor concentration that corresponded to a p-value of 0.5 on the sigmoid curve. Graphical representation of concentration was shown on a logarithmic scale to better represent and avoid skewing of data.

### Example 4

### Inter-Rater Reliability

To determine inter-rater reliability, each participant's data was first split into their first and second administrations of each odor. The reliability statistic Cronbach's alpha (α) was then computed to determine the reliability of participant yes/no responses. Additionally, the reliability of the response-bias (c) and estimated threshold concentrations for each odor were calculated. As an additional measure of reliability, the correlation coefficient Pearson's r was also determined for each measure.

Calculations of sensitivity, response bias, threshold, and reliability for the combination of all participant administrations represent mean normative data for the specific population tested. Demographics collected from each participant were analyzed for the existence of relationships with all calculated measures.

### Results of Examples

Yes-no data for all 33 subjects taking the WUTC were combined to give single measures of mean response-bias and threshold for each of the five odors administered. These statistics are presented in Table 4.

Alternatively, estimated thresholds were calculated for each individual participant and then used to provide means and standard deviations for each odor (Table 5). Mean estimated threshold values differ slightly from those found in Table 4 due to some individual estimated thresholds being too high or low to be discernible by the test and are therefore calculated from a lower number of participants. Ethanol (M=251.808, SD=246.041), Pinene (M=251.225, SD=248.933), and Vanillin (M=119.978, SD=105.570) had highest estimated threshold concentrations with those of isoamyl acetate (M=13.935, SD=16.790) and para-cresol (M=1.340, SD=1.645) being considerably lower. However, due to differences in odor strengths, threshold values were expected to differ. Standard deviations for each estimated odor threshold were also expectedly varied as concentration ranges were different for each odorant. Estimated thresholds for ethanol and para-cresol were at or within literature values. However, the estimated isoamyl acetate threshold was below its literature value and vanillin and pinene were greater than values found.

The sensitivity measures d′ and A′ were calculated from all participant data for each concentration of each odor. Each statistic is the mean across all participant trial for that odor concentration. Values for d′ are listed in Table 6 and A′ values are found in Table 7.

The reliability between calculated test measures for each test half was assessed with the use of Cronbach's alpha (α) and linear regression (r). The results of the analyses are presented in Tables 8 and 9. The measure of response-bias (c) for each odor had high reliability as measured by Cronbach's alpha and were significantly correlated at p<0.01. Both vanillin and para-cresol estimated threshold values for each test half had high levels of reliability with α=0.750 and α=0.856, respectively. Their estimates thresholds were also significantly correlated for vanillin (r=0.623) and para-cresol (r=0.749) at p<0.01. Estimated thresholds between test halves for isoamyl acetate, pinene, and ethanol were found to have low reliability as measured by Cronbach's alpha and regression analysis showed that correlations were not significant as well.

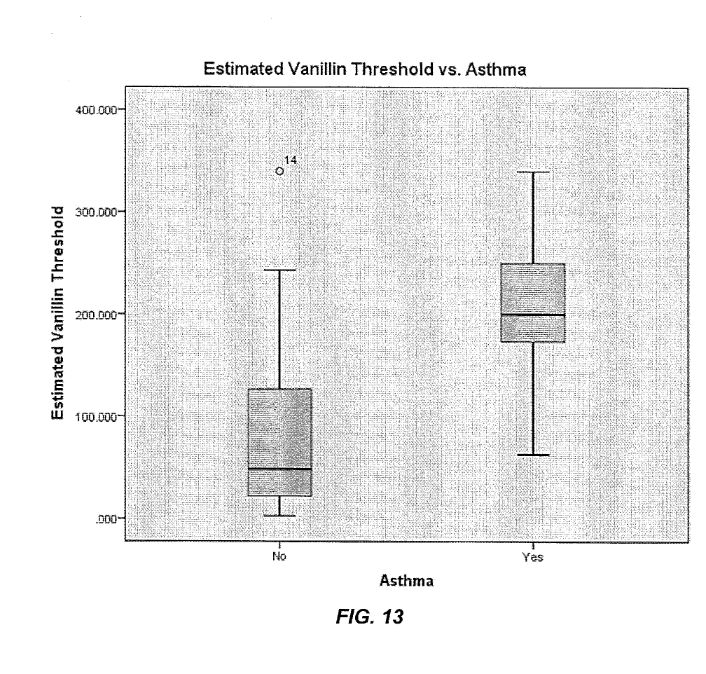

Demographics data were analyzed to determine if any significant correlations existed between them and measures of sensitivity, response bias, and estimated threshold for each odor. Mean estimated vanillin threshold values were compared between those with (N=8, M=207.137, 207.137) and without (N=18, M=86.084, SD=86.084) asthma. Those without asthma were found to have significantly lower (p<0.01) vanillin odor thresholds than those with asthma. This relationship is presented in FIG. 13).

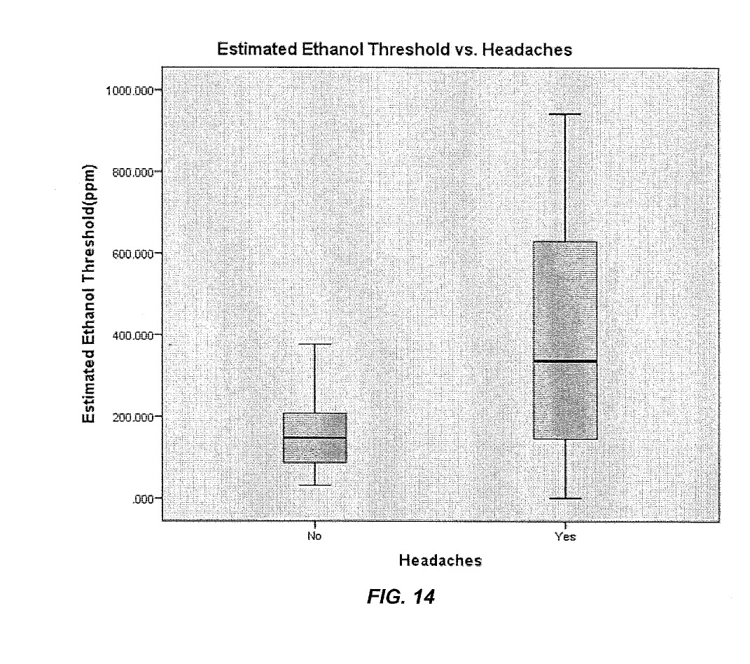

A significant difference in means was also found to exist between estimated ethanol thresholds based on subject self-report of headaches (FIG. 14). Participants with (N=8, M=396.631, SD=332.365) headaches were found to have significantly higher ethanol thresholds than those without (N=12, M=155.260, SD=94.039) headaches. This relationship was significant at p<0.05.

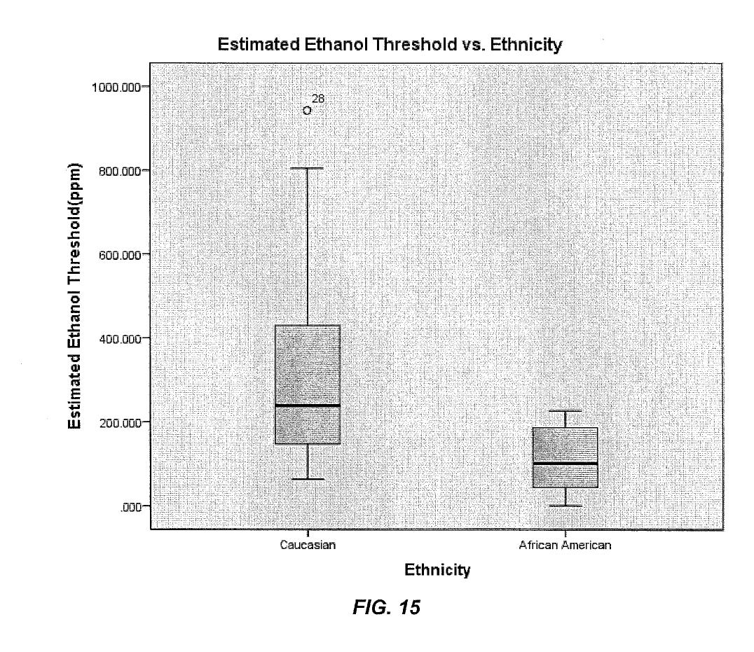

A significant relationship was also found to exist between ethnicity and mean estimated threshold values for ethanol. Caucasians were found to have higher thresholds for ethanol (M=344.968, SD=276.694) than African Americans (M=112.069, SD=82.529) significant at p<0.05. The relationship can be seen in FIG. 15.

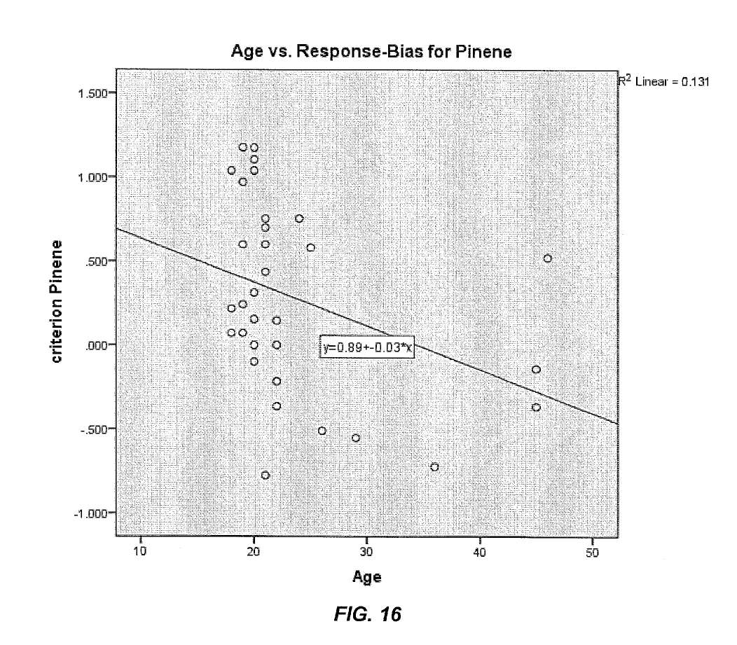

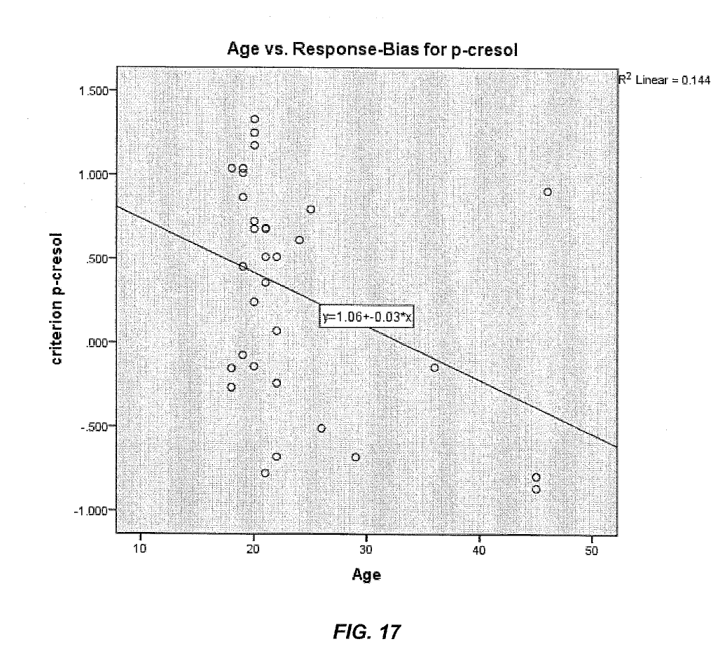

Response bias for both para-cresol and pinene were found to be significantly related to age (see FIGS. 16 and 17). For each odor, bias to respond “yes” was found to increase significantly as participants' age increased. The relationship was significant for pinene at p<0.05 (t=−2.125) and for para-cresol at p<0.05 (t=−2.250) as well.

TABLE 4
Statistical Measures of Mean Response-
Bias and Threshold for Combined Trials

| | | Threshold |
|---|---|---|
| Odorant | C | (ppm) |
| Ethanol | 0.198 | 263.750 |
| Isoamyl Acetate | 0.31 | 38.274 |
| para-cresol | 0.217 | 1.058 |
| Pinene | 0.194 | 275.598 |
| Vanillin | 0.381 | 112.426 |

TABLE 5
Descriptive Statistics for Odorant Thresholds

| | | | | | Std. |
|---|---|---|---|---|---|
| Odorant | N | Minimum | Maximum | Mean | Deviation |
| Ethanol | 20 | 1.057 | 942.747 | 251.808 | 246.041 |
| Isoamyl Acetate | 22 | 0.513 | 65.102 | 13.935 | 16.790 |
| para-cresol | 26 | 0.005 | 5.28 | 1.340 | 1.645 |
| Pinene | 24 | 0.778 | 795.338 | 251.225 | 248.933 |
| Vanillin | 25 | 2.619 | 339.932 | 19.978 | 105.570 |

TABLE 6
Mean Odorant d′ Values at Each Concentration for Combined Participant Trial

| | Lowest | | | | Concentration | | | | Highest |
|---|---|---|---|---|---|---|---|---|---|
| Odorant | 1 | 2 | 3 | 4 | 5 | 6 | 7 | 8 | 9 |
| Ethanol | 0.898 | 0.704 | 0.035 | 0.819 | 0.16 | 0.941 | 0.513 | 1.066 | 1.154 |
| Isoamyl | −0.249 | −0.198 | −0.198 | −0.053 | 0.704 | 0.704 | 1.11 | 1.299 | 1.024 |
| Acetate | | | | | | | | | |
| para-cresol | −0.416 | 0.16 | 0.32 | 0.32 | 0.475 | 1.154 | 1.458 | 1.403 | 2.246 |
| Pinene | −0.249 | 0.898 | 0.16 | 0.035 | 0.513 | 0.742 | 1.458 | 2.097 | 1.72 |
| Vanillin | −0.302 | −0.053 | 0.077 | 0.359 | 0.16 | 0.55 | 1.024 | 1.11 | 1.885 |

TABLE 7
Mean Odorant A′ Values at Each Concentration for Combined Participant Trials

| | Lowest | | | | Concentration | | | | Highest |
|---|---|---|---|---|---|---|---|---|---|
| Odorant | 1 | 2 | 3 | 4 | 5 | 6 | 7 | 8 | 9 |
| Ethanol | 0.757 | 0.716 | 0.514 | 0.741 | 0.561 | 0.766 | 0.668 | 0.789 | 0.804 |
| Isoamyl | 0.407 | 0.424 | 0.424 | 0.478 | 0.716 | 0.716 | 0.796 | 0.826 | 0.781 |
| Acetate | | | | | | | | | |
| para-cresol | 0.356 | 0.561 | 0.614 | 0.614 | 0.313 | 0.804 | 0.847 | 0.84 | 0.908 |
| Pinene | 0.407 | 0.757 | 0.561 | 0.514 | 0.668 | 0.724 | 0.847 | 0.901 | 0.875 |
| Vanillin | 0.39 | 0.478 | 0.531 | 0.625 | 0.561 | 0.678 | 0.781 | 0.796 | 0.888 |

TABLE 8
Reliability Measure between Test Halves (Cronbach's α)

| | | Threshold |
|---|---|---|
| Odorant | c | (ppm) |
| Ethanol | 0.917 | 0.299 (N = 17) |
| Isoamyl Acetate | 0.972 | 0.182 (N = 18) |
| para-cresol | 0.965 | 0.856 (N = 22) |
| Pinene | 0.945 | 0.06 (N = 22) |
| Vanillin | 0.971 | 0.750 (N = 23) |

TABLE 9
Correlation between Test Halves (Pearson's r)

| | | | Threshold |
|---|---|---|---|
| Odorant | yes-no | C | (ppm) |
| Ethanol | .423* | .865* | .19 (N = 17) |
| Isoamyl Acetate | .547* | .946* | .105 (N = 18) |
| para-cresol | .585* | .932* | .749 (N = 22)* |
| Pinene | .539* | .908* | .031 (N = 22) |
| Vanillin | .542* | .945* | .623 (N = 23)* |

*p < .01

### Discussion of Examples

Disclosed herein in some embodiments is the development of a methodology of testing participants' ability to detect multiple odors. Each odor was chosen to provide a robust and varied odor profile so that more specific relationships between disease and olfaction can be explored. With this in mind, the physiological interactions between binding proteins and odor transport, odor molecule classification, and scent type were incorporated into the development of the WUTC Threshold Test.

Along with the creation of the test, an initial study was completed with N=33 participants to observe the inter-rater reliability for measures of response bias and estimated threshold for each odor. The five odorants para-cresol, ethanol, isoamyl acetate, α-pinene, and vanillin were administered in the test alongside null-stimulus trials which made these multiple measures possible. Vanillin and para-cresol thresholds were found to be reliable (p<0.01 and α=0.750 and 0.856, respectively). While it is not desired to be bound by any particular theory of operation, this could be due to both odors having aromatic structures that are very different from those of the other odors included in the test. This similarity in structure may cause the odors to be processed similarly by the olfactory system, leading to high reliability for both. The aromatic nature of the odorants may also cause them to have higher stability, allowing more of the molecule to reach the olfactory sensory than the other odors used in the WUTC Threshold Test. While it is not desired to be bound by any particular theory of operation and as non-limiting examples, reliability for odorants can be impacted by variations in test methodology, concentration ranges (e.g. an inappropriate concentration range might lead to higher levels of participant uncertainty as to whether a stimulus was detected), or the molecular structure of the odorants themselves. Thus, these and other aspects can be considered in providing a particular test.

By combining all participant trials for each odor, the mean sensitivity for each odor concentration was obtained. These values of d′ and A′ can exhibit a pattern of increasing as the concentration of the odorant increases. This signifies an increase in the sensitivity, or ability to detect, an odor stimulus as the magnitude of the stimulus increases. However, this pattern is not seen across all odors. While it is not desired to be bound by any particular theory of operation, this may be due to the randomized nature of the trials or to differences in how each odor interacts with the olfactory system. Vanillin and para-cresol have the most consistent pattern of sensitivity increase as concentration increases. This is reflected by their high reliability across test halves. The relationship between inter-rater reliability and odor concentration is investigated further to further develop olfactory testing methodology.

Participants taking part in this study provided answers to a demographic form which made correlational analyses possible. Among the data analyzed, correlations were found to exist between response-bias for both para-cresol and pinene and participant age (p<0.05). Since the relationship was negative, the tendency of a participant to guess “yes” to a trial increased with age. While it is not desired to be bound by any particular theory of operation, this may be explained by the known decrease of olfactory ability as age increases. Additional information is obtained with a larger and more varied sample pool in regards to participant age.

An analysis between ethanol threshold and headaches found a significant interaction (p<0.05). Those with headaches had higher thresholds than those without. While it is not desired to be bound by any particular theory of operation, due to ethanol being a hydrophilic molecule, it may be possible that it encounters difficulty crossing the mucus membrane surrounding the nasal epithelium when a common cause of headaches, dehydration, occurs.

Ethanol was also found to be correlated with ethnicity with Africans Americas having significantly lower mean ethanol thresholds than Caucasians (p<0.05). While it is not desired to be bound by any particular theory of operation, a possible reason for this may be related to small differences in the olfactory system of Caucasians and African Americans that lead to higher levels of ethanol reaching the olfactory receptors.

A final significant interaction (p<0.01) was found to exist between mean estimated vanillin thresholds and a self-report of asthma. Participants with asthma had significantly higher thresholds than those without the disease. While it is not desired to be bound by any particular theory of operation, this may be due to lowered levels of airflow in those with asthma that leads to less of the odorant reaching the nasal epithelium. Because a similar relationship is not present between asthma and the other odors in the test, without being bound by any particular theory of operation the specific deficit to vanillin threshold may be due to the chemical properties of vanillin or the way that it interacts with the olfactory system.

Interestingly, there was a lack of significant interaction between participant threshold values based on gender despite previous research supporting gender differences in olfaction. While it is not desired to be bound by any particular theory of operation, this may be due to the nature of the test measuring only a small subsection of olfactory ability.

The test employed the odors ethanol, pinene, para-cresol, vanillin, and isoamyl acetate. However, these examples can be expanded on by adding or replacing odors on the test believed to be better linked to specific diseases than those used. Odors selected based on theories of evolutionary survival or social functioning could be used to search for differences in detection. The adaptable nature and developed methodology of the presently disclosed subject matter allows for multiple tests to be made to test particular populations as new links to olfactory deficits are discovered.

With the randomized presentation of odors in the WUTC, the possibility exists that there are cross-effects between odorants. Analyses are completed to determine if any relationship exists between the order of the odor trials and olfactory ability. Doing so leads to information that further enhances the usability of the test.

There were also several relationships found between the individual measures determinable by the WUTC and demographic data. These olfactory deficits were found to exist not with all odors but with particular odors used in the test. This further strengthens the argument that tests of olfaction benefit from the use of diverse stimuli as diseases might not cause global deficits to all types of odors.

The development of the methodology behind the Wheeler UTC Threshold Test represents a shift in olfactory testing archetypes. By including double-blind testing, randomization of stimuli, and multiple presentations of each odor, the WUTC conforms to standards of research methodology more than other, currently available tests. Additionally, the WUTC adopts a paradigm that relates to the nature of the stimulus. Odors were chosen for molecular diversity and differences in how they interact with the olfactory system. By using this kind of odor profile, complex relationships between olfactory ability for specific odors and certain diseases can be identified. In doing so the presently disclosed subject matter provides a valid predictor of particular ailments within individuals, an accomplishment that no other test of olfaction can do at this time.

### REFERENCES

Ames, B. N., Shigenaga, M. K., & Hagen, T. M. (1993). Oxidants, antioxidants, and the degenerative diseases of aging. Proc Natl Acad Sci USA, 90(17), 7915-7922.
Banks, W. P. (1970). Signal detection theory and human memory. Psychological bulletin, 74(2), 81.
Brand, G. (2006). Olfactory/trigeminal interactions in nasal chemoreception. Neuroscience & Biobehavioral Reviews Neuroscience & Biobehavioral Reviews, 30(7), 908-917.
Cronbach, L. J. (1951). Coefficient alpha and the internal structure of tests. Psychometrika, 16(3), 297-334.
Darren, G., & Mallery, P. (2003). SPSS for Windows step by step: a simple guide and reference, 11.0 update: Allyn and Bacon.
Deems, D. A., Doty, R. L., Settle, R. G., Moore-Gillon, V., Shaman, P., Mester, A. F., . . . Snow, J. B., Jr. (1991). Smell and taste disorders, a study of 750 patients from the University of Pennsylvania Smell and Taste Center. Arch Otolaryngol Head Neck Surg, 117(5), 519-528.
Duncan, R., & Briggs, M. (1962). Treatment of uncomplicated anosmia by vitamin A. Archives of otolaryngology (Chicago, Ill.: 1960), 75, 116-124.
Edraki, M., Pourpulad, H., Kargar, M., Pishva, N., Zare, N., & Montaseri, H. (2013). Olfactory stimulation by vanillin prevents apnea in premature newborn infants. Iranian journal of pediatrics, 23(3), 261-268.
Evola, M., Hall, A., Wall, T., Young, A., & Grammas, P. (2010). Oxidative stress impairs learning and memory in apoE knockout mice. Pharmacology Biochemistry and Behavior Pharmacology Biochemistry and Behavior, 96(2), 181-186.
Halliwell, B. (1992). Reactive Oxygen Species and the Central Nervous System. JNC Journal of Neurochemistry, 59(5), 1609-1623.
Heeger, D. (1998). Signal detection theory handout. from http://www-psych.stanford.edu/˜lera/psych115s/notes/signal/
Heeger, D. (2003). Signal detection theory. from http://www.cns.nyu.edu/˜david/handouts/sdt/sdt.html
Himmelfarb, J., Stenvinkel, P., Ikizler, T. A., & Hakim, R. M. (2002). The elephant in uremia: Oxidant stress as a unifying concept of cardiovascular disease in uremia.
Hummel, T., Sekinger, B., Wolf, S. R., Pauli, E., & Kobal, G. (1997). ‘Sniffin' sticks’: olfactory performance assessed by the combined testing of odor identification, odor discrimination and olfactory threshold. Chemical senses, 22(1), 39-52.
Ingham, J. G. (1970). Individual differences in signal detection. Acta psychologica, 34(1), 39-50.
Kasanen, J., Pasanen, A. L., Pasanen, P., Liesivuori, J., Kosma, V. M., & Alarie, Y. (1998). Stereospecificity of the sensory irritation receptor for nonreactive chemicals illustrated by pinene enantiomers. Archives of toxicology, 72(8).
Kershaw, C. D. (1985). Statistical properties of staircase estimates from two interval forced choice experiments. BMSP British Journal of Mathematical and Statistical Psychology, 38(1), 35-43.
Lavin, J., Meen, E., Lam, K., Kato, A., Huang, H., Norton, J., . . . Tan, B. (2013). The Impact and Nature of Inflammation in the Olfactory Cleft On Olfaction in Patients with Chronic Rhinosinusitis. Journal of Allergy and Clinical Immunology, 131(2, Supplement), AB59.
Lopez, I., Acuna, D., Beltran-Parrazal, L., Lopez, I. E., Amarnani, A., Cortes, M., & Edmond, J. (2009). Evidence for oxidative stress in the developing cerebellum of the rat after chronic mild carbon monoxide exposure (0.0025% in air). BMC neuroscience, 10.
MacCaffrey, T. V. (1997). Rhinologic diagnosis and treatment: Thieme.
Macmillan, N., & Kaplan, H. L. (1985). Detection theory analysis of group data: estimating sensitivity from average hit and false-alarm rates. Psychological bulletin, 98(1), 185-199.
Macmillan, N. A. (1993). Signal detection theory as data analysis method and psychological decision model. A handbook for data analysis in the behavioral sciences: Methodological issues, 21-57.
Macmillan, N. A., & Creelman, C. D. (1990). Response bias: Characteristics of detection theory, threshold theory, and “nonparametric” indexes. Psychological bulletin, 107(3), 401.
Macmillan, N. A., & Creelman, C. D. (1996). Triangles in ROC Space: History and Theory of “Nonparametric” Measures of Sensitivity and Response Bias. Psychonomic bulletin & review., 3(2), 164.
Macmillan, N. A., & Creelman, C. D. (2004). Detection Theory: A User's Guide: Taylor & Francis.
Miller, J. (1996). The sampling distribution of d′. Perception & psychophysics, 58(1), 65-72.
Misonou, H., Morishima-Kawashima, M., & Ihara, Y. (2000). Oxidative stress induces intracellular accumulation of amyloid beta-protein (Abeta) in human neuroblastoma cells. Biochemistry, 39(23), 6951-6959.
Murray, M. (2013). Our chemical senses: olfaction. http://faculty.washington.edu/chudler/chems.html
Nunnally, J. C. (1967). Psychometric theory: McGraw-Hill.
Perry, G., Cash, A. D., & Smith, M. A. (2002). Alzheimer Disease and Oxidative Stress. Journal of Biomedicine and Biotechnology Journal of Biomedicine and Biotechnology, 2(3), 120-123.
Pevsner, J., & Snyder, S. H. Odorant-binding protein: odorant transport function in the vertebrate nasal epithelium. 15(Oxford University Press), 217-222.
Reutens, S., & Sachdev, P. (2002). Homocysteine in neuropsychiatric disorders of the elderly. Int J Geriatr Psychiatry, 17(9), 859-864.
Ruff, R. L., Ruff, S. S., & Wang, X.-F. (2008). Headaches among Operation Iraqi Freedom/Operation Enduring Freedom veterans with mild traumatic brain injury associated with exposures to explosions. J Rehabil Res Dev, 45(7), 941-952.
Silver, W. (1991). Physiological factors in nasal trigeminal chemoreception. Chemical senses, II, 21-37.
Silver, W. L., Mason, J., Russell, A., Michael, A., & Smeraski, C. A. (1986). Nasal trigeminal chemoreception: Responses to n-aliphatic alcohols. BRES Brain Research, 376(2), 221-229.
Snodgrass, J. G., & Corwin, J. (1988). Pragmatics of measuring recognition memory: applications to dementia and amnesia. Journal of Experimental Psychology: General, 117(1), 34.
Stanislaw, H., & Todorov, N. (1999). Calculation of signal detection theory measures. Behavior research methods, instruments, & computers, 31(1), 137-149.
Swets, J. A. (1986). Form of empirical ROCs in discrimination and diagnostic tasks: implications for theory and measurement of performance. Psychological bulletin, 99(2), 181-198.
Tritschler, H. J., & Medori, R. (1993). Mitochondrial DNA alterations as a source of human disorders. Neurology, 43(2), 280-288.
Vanholder, R., De Smet, R., Glorieux, G., Argiles, A., Baurmeister, U., Brunet, P., . . . Deppisch, R. (2003). Review on uremic toxins: classification, concentration, and interindividual variability. Kidney international, 63(5), 1934-1943.
Vogt, R., Prestwich, G. D., & Lerner, M. R. (1991). Odorant-binding-protein subfamilies associate with distinct classes of olfactory receptor neurons in insects. Journal of neurobiology, 22(1), 74-84.
Walton, B., & Maeso, P. (2012). can you smell that? anatomy and physiology of smell. from Walton, B., & Patricia, M. (Jan., 2012 30). Can you smell that? anatomy and physiology of smell. Retrieved from https://www.google.com/url?sa=t&rct=j&q=&esrc=s&source=web&cd=1& ved=0CCwQFjAA&url=http://www.utmb.edu/otoref/grnds/anosmia-Walton-201-0130/smell-pic-2012-0130.ppt&ei=9WltUv7aMszQkQe_{—}74G4AQ&usg=AFQjCNFSIYy1mKBYk m4-nVWthTqEal7pug&sig2=ou8mDzYtRmN-NcbT8QVoVA&bvm=bv.55123115,d.eW0
Wu, D., & Cederbaum, A. I. (2003). Alcohol, oxidative stress, and free radical damage. Alcohol research & health: the journal of the National Institute on Alcohol Abuse and Alcoholism, 27(4), 277-284.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of detecting an odor threshold, the method comprising:
(a) providing two or more odors;
(b) exposing the two or more odors to a subject; and
(c) detecting an odor threshold by detecting a response in the subject to the two or more odors.

2. The method of claim 1, where the two or more odors is five odors.

3. The method of claim 1, wherein the odors are selected based on differences in how they interact with the olfactory system of the subject and/or chemical properties they possess.

4. The method of claim 1, wherein the two or more odors differ in a characteristic selected from the group consisting of a descriptive quality, a molecular classification of odorant, a level of hydrophobicity of odorant, and any combination thereof.

5. The method of claim 1, comprising presenting the two or more odors to the subject in a random sequence, one or more different concentrations, or combinations thereof.

6. The method of claim 1, comprising employing randomized, multiple presentations of odorants, optionally with null-stimulus trials.

7. The method of claim 1, comprising employing a double-blind design in exposing the two or more odors to the subject

8. The method of claim 7, comprising repeating the exposing of each odor to the subject.

9. The method of claim 1, comprising drawing a conclusion about the subject based on the odor threshold.

10. The method of claim 9, wherein the conclusion relates to a medical condition of the subject.

11. A kit for determining an odor threshold in a subject, the kit comprising:
(a) two or more containers, each container containing a different odorant composition; and
(b) instructions for determining an odor threshold by detecting a response in the subject to the two or more odors.

12. The kit of claim 11, comprising five containers, each containing a different odorant composition.

13. The kit of claim 11, wherein the odors are selected based on differences in how they interact with the olfactory system of the subject and/or one or more chemical properties the odorant compositions possess.

14. The kit of claim 11, wherein the two or more odors differ in a characteristic selected from the group consisting of a descriptive quality, a molecular classification of odorant, a level of hydrophobicity of odorant, and any combination thereof.

15. The kit of claim 11, wherein the instructions include presenting the two or more odors to the subject in a random sequence.

16. The kit of claim 11, wherein the instructions include comprising employing randomized, multiple presentations of odorants, optionally with null-stimulus trials.

17. The kit of claim 11, wherein the instructions include employing a double-blind design in exposing the two or more odors to the subject.

18. The kit of claim 11, wherein the instructions include repeating the exposing of each odor to the subject.

19. The kit of claim 11, wherein the instructions include drawing a conclusion about the subject based on the odor threshold.

20. The kit of claim 19, wherein the conclusion relates to a medical condition of the subject.
